(19) United States
(12) Patent Application Publication
(10) Pub. No.: US 2007/0141714 A1
(43) Pub. Date: Jun. 21, 2007

(54) Method to detect small molecules binding to proteins using surface enhanced Raman scattering (SERS)

(75) Inventors: Kung-bin Sung, Seattle, WA (US); Narayan Sundararajan, San Francisco, CA (US); Selena Chan, Sunnyvale, CA (US)

Correspondence Address: MORRISON & FOERSTER LLP, 1650 TYSONS BOULEVARD, SUITE 400, MCLEAN, VA, 22102, US

(73) Assignee: Intel Corporation, Santa Clara, CA (US)

(21) Appl. No.: 11/305,340

(22) Filed: Dec. 19, 2005

(51) Int. Cl. G01N 33/20

(52) U.S. Cl. 436/73

(57) ABSTRACT

Embodiments of the invention relate to detecting binding of a first analyte to a second analyte by Raman spectroscopy. An embodiment includes attaching one analyte to a substrate and then detecting the binding of another analyte to the analyte on the substrate by Raman spectroscopy. Another embodiment includes contacting analytes in a fluid and then detecting the binding of one analyte to another analyte by Raman spectroscopy.

### FIELD OF THE INVENTION

The embodiments of the invention relate to detecting binding of a first analyte to a second analyte by Raman spectroscopy. These tools and methods can be used, for example, to detect interaction between small molecule analytes and protein analytes. The invention transcends several scientific disciplines such as polymer chemistry, biochemistry, molecular biology, medicine and medical diagnostics.

### BACKGROUND

Raman spectroscopy is one analytical technique that provides rich optical-spectral information, and surface-enhanced Raman spectroscopy (SERS) has proven to be one of the most sensitive methods for performing quantitative and qualitative analyses. A Raman spectrum, similar to an infrared spectrum, consists of a wavelength distribution of bands corresponding to molecular vibrations specific to the sample being analyzed (the analyte). In the practice of Raman spectroscopy, the beam from a light source, generally a laser, is focused upon the sample to thereby generate inelastically scattered radiation, which is optically collected and directed into a wavelength-dispersive spectrometer in which a detector converts the energy of impinging photons to electrical signal intensity.

Among many analytical techniques that can be used for chemical structure analysis, Raman spectroscopy is attractive for its capability to provide rich structure information from a small optically-focused area or detection cavity. Compared to a fluorescent spectrum that normally has a single peak with half peak width of tens of nanometers to hundreds of nanometers, a Raman spectrum has multiple bonding-structure-related peaks with half peak width of as small as a few nanometers.

Although Raman spectroscopy has proven effective for identifying certain compounds, up till now, identifying interactions between compounds has not proven successful. Further, the ability to detect interactions between small molecules and proteins has becoming increasingly important as we learn that detecting these interactions can be useful in developing new pharmaceutical treatments.

### BRIEF DESCRIPTION OF THE DRAWINGS

FIG. 1 shows a schematic of an embodiment for detecting the binding of small molecules to proteins immobilized on a substrate surface.

FIG. 2 shows a schematic of an embodiment for detecting the binding of small molecules to proteins in which the proteins are mixed in a fluid.

FIG. 3 is a diagram of the Raman spectra produced by the process described in FIG. 2.

### DETAILED DESCRIPTION

As used in the specification and claims, the singular forms “a”, “an” and “the” include plural references unless the context clearly dictates otherwise. For example, the term “an array” may include a plurality of arrays unless the context clearly dictates otherwise.

An “array,” “macroarray” or “microarray” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the sample spots on the array. A macroarray generally contains sample spot sizes of about 300 microns or larger and can be easily imaged by gel and blot scanners. A microarray would generally contain spot sizes of less than 300 microns. A multiple-well array is a support that includes multiple chambers for containing sample spots.

“Solid support,” “support,” and “substrate” refer to a material or group of materials having a rigid or semi-rigid surface or surfaces. In some aspects, at least one surface of the solid support will be substantially flat, although in some aspects it may be desirable to physically separate synthesis regions for different molecules with, for example, wells, raised regions, pins, etched trenches, or the like. In certain aspects, the solid support(s) will take the form of beads, resins, gels, microspheres, or other geometric configurations.

The term “analyte”, “target” or “target molecule” refers to a molecule of interest that is to be analyzed and can be any molecule or compound. The analyte may be a Raman active compound or a Raman inactive compound. Further, the analyte could be an organic or inorganic molecule. Some examples of analytes may include a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to molecular probes such as chemically modified carbon nanotubes, carbon nanotube bundles, nanowires, nanoclusters or nanoparticles. The analyte molecule may be fluorescently labeled DNA or RNA.

An analyte can be in the solid, liquid, gaseous or vapor phase. By “gaseous or vapor phase analyte” is meant a molecule or compound that is present, for example, in the headspace of a liquid, in ambient air, in a breath sample, in a gas, or as a contaminant in any of the foregoing. It will be recognized that the physical state of the gas or vapor phase can be changed by pressure, temperature as well as by affecting surface tension of a liquid by the presence of or addition of salts etc.

The analyte can be comprised of a member of a specific binding pair (sbp) and may be a ligand, which is monovalent (monoepitopic) or polyvalent (polyepitopic), usually antigenic or haptenic, and is a single compound or plurality of compounds which share at least one common epitopic or determinant site. The analyte can be a part of a cell such as bacteria or a cell bearing a blood group antigen such as A, B, D, etc., or an HLA antigen or a microorganism, e.g., bacterium, fungus, protozoan, or virus. In certain aspects of the invention, the analyte is charged.

A member of a specific binding pair (“sbp member”) is one of two different molecules, having an area on the surface or in a cavity which specifically binds to and is thereby defined as complementary with a particular spatial and polar organization of the other molecule. The members of the specific binding pair are referred to as ligand and receptor (antiligand) or analyte and probe. Therefore, a probe is a molecule that specifically binds an analyte. These will usually be members of an immunological pair such as antigen-antibody, although other specific binding pairs such as biotin-avidin, hormones-hormone receptors, nucleic acid duplexes, IgG-protein A, polynucleotide pairs such as DNA-DNA, DNA-RNA, and the like are not immunological pairs but are included in the invention and the definition of sbp member.

Specific binding is the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide hybridization interactions, and so forth.

Non-specific binding is non-covalent binding between molecules that is relatively independent of specific surface structures. Non-specific binding may result from several factors including hydrophobic interactions between molecules.

The methods of the present invention may be used to detect the presence of a particular target analyte, for example, a nucleic acid, oligonucleotide, protein, enzyme, antibody or antigen. The methods may also be used to screen bioactive agents, i.e. drug candidates, for binding to a particular target or to detect agents like pollutants.

The polyvalent ligand analytes will normally be poly(amino acids), i.e. polypeptides and proteins, polysaccharides, nucleic acids, and combinations thereof. Such combinations include components of bacteria, viruses, chromosomes, genes, mitochondria, nuclei, cell membranes and the like.

For the most part, the polyepitopic ligand analytes to which the subject invention can be applied will have a molecular weight of at least about 5,000, more usually at least about 10,000. In the poly(amino acid) category, the poly(amino acids) of interest will generally be from about 5,000 to 5,000,000 molecular weight, more usually from about 20,000 to 1,000,000 molecular weight; among the hormones of interest, the molecular weights will usually range from about 5,000 to 60,000 molecular weight.

The monoepitopic ligand analytes will generally be from about 100 to 2,000 molecular weight, more usually from 125 to 1,000 molecular weight. The analytes include drugs, metabolites, pesticides, pollutants, and the like. Included among drugs of interest are the alkaloids. Among the alkaloids are morphine alkaloids, which includes morphine, codeine, heroin, dextromethorphan, their derivatives and metabolites; cocaine alkaloids, which include cocaine and benzyl ecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites.

The term analyte further includes polynucleotide analytes such as those polynucleotides defined below. These include m-RNA, r-RNA, t-RNA, DNA, DNA-RNA duplexes, etc. The term analyte also includes receptors that are polynucleotide binding agents, such as, for example, peptide nucleic acids (PNA), restriction enzymes, activators, repressors, nucleases, polymerases, histones, repair enzymes, chemotherapeutic agents, and the like.

The analyte may be a molecule found directly in a sample such as a body fluid from a host. The sample can be examined directly or may be pretreated to render the analyte more readily detectible. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

The term “probe” or “probe molecule” refers to a molecule that binds to a target molecule for the analysis of the target. The probe or probe molecule is generally, but not necessarily, has a known molecular structure or sequence. The probe or probe molecule is generally, but not necessarily, attached to the substrate of the array. The probe or probe molecule is typically a nucleotide, an oligonucleotide, or a protein, including, for example, cDNA or pre-synthesized polynucleotide deposited on the array. Probes molecules are biomolecules capable of undergoing binding or molecular recognition events with target molecules. (In some references, the terms “target” and “probe” are defined opposite to the definitions provided here.) The polynucleotide probes require only the sequence information of genes, and thereby can exploit the genome sequences of an organism. In cDNA arrays, there could be cross-hybridization due to sequence homologies among members of a gene family. Polynucleotide arrays can be specifically designed to differentiate between highly homologous members of a gene family as well as spliced forms of the same gene (exon-specific). Polynucleotide arrays of the embodiment of this invention could also be designed to allow detection of mutations and single nucleotide polymorphism. A probe or probe molecule can be a capture molecule.

The term “bi-functional linker group” refers to an organic chemical compound that has at least two chemical groups or moieties, such are, carboxyl group, amine group, thiol group, aldehyde group, epoxy group, that can be covalently modified specifically; the distance between these groups is equivalent to or greater than 5-carbon bonds.

The term “capture molecule” refers to a molecule that is immobilized on a surface. The capture molecule is generally, but not necessarily, binds to a target or target molecule. The capture molecule is typically a nucleotide, an oligonucleotide, or a protein, but could also be a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to a target molecule that is bound to a probe molecule to form a complex of the capture molecule, target molecule and the probe molecule. The capture molecule may be fluorescently labeled DNA or RNA. The capture molecule may or may not be capable of binding to just the target molecule or just the probe molecule.

The term “molecule” generally refers to a macromolecule or polymer as described herein. However, arrays comprising single molecules, as opposed to invention.

“Predefined region” or “spot” or “pad” refers to a localized area on a solid support. The spot could be intended to be used for formation of a selected molecule and is otherwise referred to herein in the alternative as a “selected” region. The spot may have any convenient shape, e.g., circular, rectangular, elliptical, wedge-shaped, etc. For the sake of brevity herein, “predefined regions” are sometimes referred to simply as “regions” or “spots.” In some embodiments, a predefined region and, therefore, the area upon which each distinct molecule is synthesized is smaller than about 1 cm² or less than 1 mm², and still more preferably less than 0.5 mm². In most preferred embodiments the regions have an area less than about 10,000 μm² or, more preferably, less than 100 μm², and even more preferably less than 10 μm² or less than 1 μm². Additionally, multiple copies of the polymer will typically be synthesized within any preselected region. The number of copies can be in the hundreds to the millions. A spot could contain an electrode to generate an electrochemical reagent, a working electrode to synthesize a polymer and a confinement electrode to confine the generated electrochemical reagent. The electrode to generate the electrochemical reagent could be of any shape, including, for example, circular, flat disk shaped and hemisphere shaped.

The term “nucleotide” includes deoxynucleotides and analogs thereof. These analogs are those molecules having some structural features in common with a naturally occurring nucleotide such that when incorporated into a polynucleotide sequence, they allow hybridization with a complementary polynucleotide in solution. Typically, these analogs are derived from naturally occurring nucleotides by replacing and/or modifying stabilize or destabilize hybrid formation, or to enhance the specificity of hybridization with a complementary polynucleotide sequence as desired, or to enhance stability of the polynucleotide.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers.

An “oligonucleotide” is a polynucleotide having 2 to 20 nucleotides. Analogs also include protected and/or modified monomers as are conventionally used in polynucleotide synthesis. As one of skill in the art is well aware, polynucleotide synthesis uses a variety of base-protected nucleoside derivatives in which one or more of the nitrogens of the purine and pyrimidine moiety are protected by groups such as dimethoxytrityl, benzyl, tert-butyl, isobutyl and the like.

For instance, structural groups are optionally added to the ribose or base of a nucleoside for incorporation into a polynucleotide, such as a methyl, propyl or allyl group at the 2′-O position on the ribose, or a fluoro group which substitutes for the 2′-O group, or a bromo group on the ribonucleoside base. 2′-O-methyloligoribonucleotides (2′-O-MeORNs) have a higher affinity for complementary polynucleotides (especially RNA) than their unmodified counterparts. Alternatively, deazapurines and deazapyrimidines in which one or more N atoms of the purine or pyrimidine heterocyclic ring are replaced by C atoms can also be used.

The phosphodiester linkage, or “sugar-phosphate backbone” of the polynucleotide can also be substituted or modified, for instance with methyl phosphonates, O-methyl phosphates or phosphororthioates. Another example of a polynucleotide comprising such modified linkages for purposes of this disclosure includes “peptide polynucleotides” in which a polyamide backbone is attached to polynucleotide bases, or modified polynucleotide bases. Peptide polynucleotides which comprise a polyamide backbone and the bases found in naturally occurring nucleotides are commercially available.

Nucleotides with modified bases can also be used in the embodiments of the invention. Some examples of base modifications include 2-aminoadenine, 5-methylcytosine, 5-(propyn-1-yl)cytosine, 5-(propyn-1-yl)uracil, 5-bromouracil, 5-bromocytosine, hydroxymethylcytosine, methyluracil, hydroxymethyluracil, and dihydroxypentyluracil which can be incorporated into polynucleotides in order to modify binding affinity for complementary polynucleotides.

Groups can also be linked to various positions on the nucleoside sugar ring or on the purine or pyrimidine rings which may stabilize the duplex by electrostatic interactions with the negatively charged phosphate backbone, or through interactions in the major and minor groves. For example, adenosine and guanosine nucleotides can be substituted at the N² position with an imidazolyl propyl group, increasing duplex stability. Universal base analogues such as 3-nitropyrrole and 5-nitroindole can also be included. A variety of modified polynucleotides suitable for use in the embodiments of the invention are described in the literature.

When the macromolecule of interest is a peptide, the amino acids can be any amino acids, including α, β, or ω-amino acids. When the amino acids are α-amino acids, either the L-optical isomer or the D-optical isomer may be used. Additionally, unnatural amino acids, for example, β-alanine, phenylglycine and homoarginine are also contemplated by the embodiments of the invention. These amino acids are well-known in the art.

A “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often more than 20 amino acid monomers long.

A “protein” is a long polymer of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies.

The term “sequence” refers to the particular ordering of monomers within a macromolecule and it may be referred to herein as the sequence of the macromolecule.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” For example, hybridization refers to the formation of hybrids between a probe polynucleotide (e.g., a polynucleotide of the invention which may include substitutions, deletion, and/or additions) and a specific target polynucleotide (e.g., an analyte polynucleotide) wherein the probe preferentially hybridizes to the specific target polynucleotide and substantially does not hybridize to polynucleotides consisting of sequences which are not substantially complementary to the target polynucleotide. However, it will be recognized by those of skill that the minimum length of a polynucleotide desired for specific hybridization to a target polynucleotide will depend on several factors: G/C content, positioning of mismatched bases (if any), degree of uniqueness of the sequence as compared to the population of target polynucleotides, and chemical nature of the polynucleotide (e.g., methylphosphonate backbone, phosphorothiolate, etc.), among others.

Methods for conducting polynucleotide hybridization assays have been well developed in the art. Hybridization assay procedures and conditions will vary depending on the application and are selected in accordance with the general binding methods known in the art.

It is appreciated that the ability of two single stranded polynucleotides to hybridize will depend upon factors such as their degree of complementarity as well as the stringency of the hybridization reaction conditions.

As used herein, “stringency” refers to the conditions of a hybridization reaction that influence the degree to which polynucleotides hybridize. Stringent conditions can be selected that allow polynucleotide duplexes to be distinguished based on their degree of mismatch. High stringency is correlated with a lower probability for the formation of a duplex containing mismatched bases. Thus, the higher the stringency, the greater the probability that two single-stranded polynucleotides, capable of forming a mismatched duplex, will remain single-stranded. Conversely, at lower stringency, the probability of formation of a mismatched duplex is increased.

The appropriate stringency that will allow selection of a perfectly-matched duplex, compared to a duplex containing one or more mismatches (or that will allow mismatch) is generally determined empirically. Means for adjusting the stringency of a hybridization reaction are well-known to those of skill in the art.

A “ligand” is a molecule that is recognized by a particular receptor. Examples of ligands that can be investigated by this invention include, but are not restricted to, agonists and antagonists for cell membrane receptors, toxins and venoms, viral epitopes, hormones, hormone receptors, peptides, enzymes, enzyme substrates, cofactors, drugs (e.g. opiates, steroids, etc.), lectins, sugars, polynucleotides, nucleic acids, oligosaccharides, proteins, and monoclonal antibodies.

A “receptor” is molecule that has an affinity for a given ligand. Receptors may-be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term “receptors” is used herein, no difference in meaning is intended. A “Ligand Receptor Pair” is formed when two macromolecules have combined through molecular recognition to form a complex. Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value would be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies: For instance, the invention may be useful in investigating the ligand-binding site on the antibody molecule which combines with the epitope of an antigen of interest; determining a sequence that mimics an antigenic epitope may lead to the-development of vaccines of which the immunogen is based on one or more of such sequences or lead to the development of related diagnostic agents or compounds useful in therapeutic treatments such as for auto-immune diseases (e.g., by blocking the binding of the “anti-self” antibodies).

d) Nucleic Acids: Sequences of nucleic acids may be synthesized to establish DNA or RNA binding sequences.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant.

f) Hormone receptors: Examples of hormones receptors include, e.g., the receptors for insulin and growth hormone. Determination of the ligands which bind with high affinity to a receptor is useful in the development of, for example, an oral replacement of the daily injections which diabetics take to relieve the symptoms of diabetes. Other examples are the vasoconstrictive hormone receptors; determination of those ligands which bind to a receptor may lead to the development of drugs to control blood pressure.

g) Opiate receptors: Determination of ligands which bind to the opiate receptors in the brain is useful in the development of less-addictive replacements for morphine and related drugs.

The phrase “SERS active particle” refers to particles that produce the surface-enhanced Raman scattering effect. The SERS active particles generate surface enhanced Raman signal specific to the analyte molecules when the analyte-particle complexes are excited with a light source as compared to the Raman signal from the analyte alone in the absence of the SERS active particles. The enhanced Raman scattering effect provides a greatly enhanced Raman signal from Raman-active analyte molecules that have been adsorbed onto certain specially-prepared SERS active particle surfaces. Typically, the SERS active particle surfaces are metal surfaces. Increases in the intensity of Raman signal have been regularly observed on the order of 10⁴-10¹⁴ for some systems. SERS active particles include a variety of metals including coinage (Au, Ag, Cu), alkalis (Li, Na, K), Al, Pd and Pt.

The term “COIN” refers to a composite-organic-inorganic nanocluster(s)/nanoparticle(s). The COIN could be surface-enhanced Raman scattering incorporated into a gel matrix and used in certain other analyte separation techniques described herein. COINs are composite organic-inorganic nanoclusters. These SERS-active probe constructs comprise a core and a surface, wherein the core comprises a metallic colloid comprising a first metal and a Raman-active organic compound. The COINs can further comprise a second metal different from the first metal, wherein the second metal forms a layer overlying the surface of the nanoparticle. The COINs can further comprise an organic layer overlying the metal layer, which organic layer comprises the probe. Suitable probes for attachment to the surface of the SERS-active nanoclusters include, without limitation, antibodies, antigens, polynucleotides, oligonucleotides, receptors, ligands, and the like.

The metal required for achieving a suitable SERS signal is inherent in the COIN, and a wide variety of Raman-active organic compounds can be incorporated into the particle. Indeed, a large number of unique Raman signatures can be created by employing nanoclusters containing Raman-active organic compounds of different structures, mixtures, and ratios. Thus, the methods described herein employing COINs are useful for the simultaneous detection of many analytes in a sample, resulting in rapid qualitative analysis of the contents of “profile” of a body fluid.

COINs could be prepared using standard metal colloid chemistry. The preparation of COINs also takes advantage of the ability of metals to adsorb organic compounds. Indeed, since Raman-active organic compounds are adsorbed onto the metal during formation of the metallic colloids, many Raman-active organic compounds can be incorporated into the COIN without requiring special attachment chemistry.

In general, the COINs could be prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. COINs of different sizes can be enriched by centrifugation.

The COINs can include a second metal different from the first metal, wherein the second metal forms a layer overlying the surface of the nanoparticle. To prepare this type of SERS-active nanoparticle, COINs are placed in an aqueous solution containing suitable second metal cations and a reducing agent. The components of the solution are then subject to conditions that reduce the second metallic cations so as to form a metallic layer overlying the surface of the nanoparticle. In certain embodiments, the second metal layer includes metals, such as, for example, silver, gold, platinum, aluminum, and the like. Typically, COINs are clustered structures and range in size from about 50 nm to 100 nm.

Typically, organic compounds are attached to a layer of a second metal in COINs by covalently attaching organic compounds to the surface of the metal layer Covalent attachment of an organic layer to the metallic layer can be achieved in a variety ways well known to those skilled in the art, such as for example, through thiol-metal bonds. In alternative embodiments, the organic molecules attached to the metal layer can

The COIN(s) can include cores containing magnetic materials, such as, for example, iron oxides, and the like such that the COIN is a magnetic COIN. Magnetic COINs can be handled without centrifugation using commonly available magnetic particle handling systems. Indeed, magnetism can be used as a mechanism for separating biological targets attached to magnetic COIN particles tagged with particular biological probes.

As used herein, “Raman-active organic compound” refers to an organic molecule that produces a unique SERS signature in response to excitation by a laser. A variety of Raman-active organic compounds are contemplated for use as components in COINs. In certain embodiments, Raman-active organic compounds are polycyclic aromatic or heteroaromatic compounds. Typically the Raman-active organic compound has a molecular weight less than about 300 Daltons.

Additional, non-limiting examples of Raman-active organic compounds useful in COINs include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins, aminoacridine, and the like.

In certain embodiments, the Raman-active compound is adenine, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, or 9-amino-acridine 4-amino-pyrazolo(3,4-d)pyrimidine, rhodamine 6G, rhodamine B, crystal violet, basic fuchsin, cyanine 2, cyanine 3, or 2-fluoroadenine. In one embodiment, the Raman-active compound is adenine.

When “fluorescent compounds” are incorporated into COINs, the fluorescent compounds can include, but are not limited to, dyes, intrinsically fluorescent proteins, lanthanide phosphors, and the like. Dyes useful for incorporation into COINs include, for example, rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS), Lucifer Yellow, IAEDANS, 7-Me₂, N-coumarin-4-acetate, 7-OH-4-CH₃-coumarin-3-acetate, 7-NH₂-4CH₃-coumarin-3-acetate (AMCA), monobromobimane, pyrene trisulfonates, such as Cascade Blue, and monobromotrimethyl-ammoniobimane.

Multiplex testing of a complex sample would generally be based on a coding system that possesses identifiers for a large number of reactants in the sample. The primary variable that determines the achievable numbers of identifiers in currently known coding systems is, however, the physical dimension. Techniques, based on surface-enhanced Raman scattering (SERS) of organic compounds, could be used in the embodiments of this invention for developing chemical structure-based coding systems. The organic compound-assisted metal fusion (OCAM) method could be used to produce composite organic-inorganic nanoparticles (COIN) that are highly effective in generating SERS signals allows synthesis of COIN labels from a wide range of organic compounds biological sample. Thus COIN particles may be used as a coding system for multiplex and amplification-free detection of bioanalytes at near single molecule levels.

COIN particles generate intrinsic SERS signal without additional reagents. Using the OCAMF-based COIN synthesis chemistry, it is possible to generate a large number of different COIN signatures by mixing a limited number of Raman labels for use in multiplex assays in different ratios and combinations. In a simplified scenario, the Raman spectrum of a sample labeled with COIN particles may be characterized by three parameters: (a) peak position (designated as L), which depends on the chemical structure of Raman labels used and the umber of available labels, (b) peak number (designated as M), which depends on the number oflabels used together in a single COIN, and (c) peak height (designated as i), which depends on the ranges of relative peak intensity.

The total number of possible distinguishable Raman signatures (designated as T) may be calculated from the following equation:

$$T = {\sum }_{k = 1}^{M} \frac{L !}{( L - k ) ! k !} P ( i , k )$$

where P(i, k)=i^{k}−i+1, being the intensity multiplier which represents the number of distinct Raman spectra that may be generated by combining k (k=1 to M) labels for a given i value. The multiple organic compounds may be mixed in various combinations, numbers and ratios to make the multiple distinguishable Raman signatures. It has been shown that spectral signatures having closely positioned peaks (15 cm⁻¹) may be resolved visually. Theoretically, over a million of Raman signatures may be made within the Raman shift range of 500-2000 cm⁻¹ by incorporating multiple organic molecules into COIN as Raman labels using the OCAMF-based COIN synthesis chemistry.

Thus, OCAMF chemistry allows incorporation of a wide range of Raman labels into metal colloids to perform parallel synthesis of a large number of COIN labels with distinguishable Raman signatures in a matter of hours by mixing several organic Raman-active compounds of different structures, mixtures, and ratios for use in the invention methods described herein.

COINs may be used to detect the presence of a particular target analyte, for example, a nucleic acid, oligonucleotide, protein, enzyme, antibody or antigen. The nanoclusters may also be used to screen bioactive agents, i.e. drug candidates, for binding to a particular target or to detect agents like pollutants. Any analyte for which a probe moiety, such as a peptide, protein, oligonucleotide or aptamer, may be designed can be used in combination with the disclosed nanoclusters.

Also, SERS-active COINs that have an antibody as binding partner could be used to detect interaction of the Raman-active antibody labeled constructs with antigens either in solution or on a solid support. It will be understood that such immunoassays can be performed using known methods such as are used, for example, in ELISA assays, Western blotting, or protein arrays, utilizing a SERS-active COIN having an antibody as the probe and acting as either a primary or a secondary antibody, in place of a primary or secondary antibody labeled with an enzyme or a radioactive compound. In another example, a SERS-active COIN is attached to an enzyme probe for use in detecting interaction of the enzyme with a substrate.

Another group of exemplary methods could use the SERS-active COINs to detect a target nucleic acid. Such a method is useful, for example, for detection of infectious agents within a clinical sample, detection of an amplification product derived from genomic DNA or RNA or message RNA, or detection of a gene (cDNA) insert within a clone. For certain methods aimed at detection of a target polynucleotide, an oligonucleotide probe is synthesized using methods known in the art. The oligonucleotide is then used to functionalize a SERS-active COIN. Detection of the specific Raman label in the SERS-active COIN identifies the nucleotide sequence of the oligonucleotide probe, which in turn provides information regarding the nucleotide sequence of the target polynucleotide.

The terms “spectrum” or “spectra” refer to the intensities of electromagnetic radiation as a function of wavelength or other equivalent units, such as wavenumber, frequency, and energy level.

The term “spectrometer” refers to an instrument equipped with scales for measuring wavelengths or indexes of refraction.

The term “fluid” used herein means an aggregate of matter that has the tendency to assume the shape of its container, for example a liquid or gas. Analytes in fluid form can include fluid suspensions and solutions of solid particle analytes.

Embodiments of the invention relate to detecting binding of a first analyte to a second analyte by Raman spectroscopy. One analyte may be attached to a substrate and then the binding of another analyte to the analyte on the substrate may be detected by Raman spectroscopy. Alternatively, the binding of the analytes may be detected in a fluid by Raman spectroscopy.

More specifically, one embodiment is a method of detecting binding of a first analyte to a second analyte. The method includes contacting a fluid including a first analyte to a second analyte attached to a substrate, determining whether there is a decrease in the concentration of the first analyte in the fluid after contacting the first analyte in fluid to the second analyte by Raman spectroscopy.

Preferably, the first analyte is a molecule with a molecular weight less than 5,000 Da. Preferably, the second analyte is a biomolecule, protein or an enzyme. Preferably, the substrate includes glass, nickel, magnetic metal, gold, silicon, nitrocellulose, or Polyvinylidene Difluoride (PVDF).

Preferably, SERS active particles are used in the Raman spectroscopy to enhance the Raman signal of the first analyte. Preferred SERS active particles include gold, silver, copper, lithium, sodium, potassium, palladium, platinum, or aluminum.

Preferably, the determination of whether there is a decrease in the concentration of the first analyte in the fluid by Raman spectroscopy includes obtaining a first Raman spectrum of the first analyte in the fluid prior to contacting the first analyte in the fluid to the second analyte, obtaining a second Raman spectrum of the first analyte in the fluid after contacting the first analyte in the fluid to the second analyte, and comparing the first Raman spectrum to the second Raman spectrum.

Another embodiment is a method of detecting binding of a first analyte to a second analyte. The method includes introducing a second analyte attached to a substrate to an environment that comprises unbound first analyte, removing the second analyte from the environment comprising unbound first analyte, subjecting the second analyte to conditions that release the first analyte bound to the second analyte, and detecting the presence of the released first analyte by Raman spectroscopy.

Preferably, the conditions that release the first analyte bound to the second analyte includes heating the substrate, denaturing the second analyte, or replacing the first analyte by competitive binding to the second analyte.

Yet another embodiment is a method of detecting binding of a first analyte to a second analyte. The method includes introducing a second analyte attached to a substrate to an environment that comprises a first analyte unbound to the second analyte, and detecting the presence of the first analyte bound to the second analyte on the substrate by Raman spectroscopy. Preferably, the method also includes removing the second analyte attached to the substrate from the environment prior to detecting the presence of the first analyte bound to the second analyte.

Another embodiment is a device for detecting binding of a first analyte to a second analyte. The device includes a first analyte in contact with a second analyte attached to a substrate and a Raman spectrometer. The Raman spectrometer detects binding of the first analyte to the second analyte attached to the substrate. The first analyte can be unbound from the second analyte prior to being detected by the Raman spectrometer; alternatively, the Raman spectrometer can detect the presence of the first analyte while the first analyte is attached to the second analyte. Preferably, the first analyte is unbound from the second analyte by heating the substrate, denaturing the second analyte, or replacing the first analyte by competitive binding to the second analyte. Preferably, the device includes SERS active particles.

Another embodiment is a method of detecting binding of a first analyte to a second analyte. The method includes contacting a first analyte to a second analyte to form a complex comprising the first analyte bound to the second analyte, separating unbound first analyte from the complex, and detecting the presence of the first analyte in the complex by Raman spectroscopy. Preferably, the unbound first analyte is separated from the complex by a process that comprises centrifugation or filtration. Preferably, the contacting the first analyte to the second analyte is performed in a fluid comprising the first analyte and the second analyte.

Preferably, the detecting of the first analyte in the complex by spectroscopy includes separating the first analyte from the complex and detecting the presence of the separated first analyte.

An additional embodiment is a method of detecting binding of a first analyte to a second analyte. The method includes contacting unbound first analyte to a second analyte to form a complex comprising the first analyte bound to the second analyte, and detecting a decrease in the concentration of the unbound first analyte after contacting the first analyte to the second analyte in the complex by Raman spectroscopy. The complex may be separated from the unbound first analyte prior to detecting a decrease in the concentration of the unbound first analyte.

It has been determined that surface-enhanced Raman scattering (SERS) can be used to detect the binding of one analyte to another. In particular, it has been found that the binding of small molecules (molecular weight less than 5,000 Da) to biomolecules, in particular proteins (such as enzymes) can be detected by SERS. This provides a new tool for interrogating small molecule-protein interaction.

In the practice of the present invention, the Raman spectrometer can be part of a detection unit designed to detect and quantify metallic colloids of the present invention by Raman spectroscopy. Methods for detection of Raman labeled analytes, for example nucleotides, using Raman spectroscopy are known in the art. Variations on surface enhanced Raman spectroscopy (SERS), surface enhanced resonance Raman spectroscopy (SERRS) and coherent anti-Stokes Raman spectroscopy (CARS) are also known and are included within the present invention.

A non-limiting example of a Raman detection includes an excitation beam that is generated by either a frequency doubled Nd:YAG laser at 532 nm wavelength or a frequency doubled Ti:sapphire laser at 365 nm wavelength. Pulsed laser beams or continuous laser beams may be used. The excitation beam passes through confocal optics and a microscope objective, and is focused onto the flow path and/or the flow-through cell. The Raman emission light from the labeled silver colloids is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector, that includes an avalanche photodiode interfaced with a computer for counting and digitization of the signal.

Another example of a Raman detection unit is includes a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm, a light emitting diode, an Nd:YLF laser, and/or various ions lasers and/or dye lasers. The excitation beam may be spectrally purified with a bandpass filter (Corion) and may be focused on the flow path and/or flow-through cell using a 6× objective lens (Newport, Model L6X). The objective lens may be used to both excite the analyte and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors may be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art may be used for detection in the methods of the present invention, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In certain aspects of the invention, a system for detecting an analyte of the present invention includes an information processing system. An exemplary information processing system may incorporate a computer that includes a bus for communicating information and a processor for processing information. In one embodiment of the invention, the processor is selected from the Pentium® family of processors, including without limitation the Pentium® II family, the Pentium® III family and the Pentium® 4 family of processors available from Intel Corp. (Santa Clara, Calif.). In alternative embodiments of the invention, the processor may be a Celeron®, an Itanium®, or a Pentium Xeon® processor (Intel Corp., Santa Clara, Calif.). In various other embodiments of the invention, the processor may be based on Intel® architecture, such as Intel® IA-32 or Intel® IA-64 architecture. Alternatively, other processors may be used. The information processing and control system may further comprise any peripheral devices known in the art, such as memory, display, keyboard and/or other devices.

In particular examples, the detection unit can be operably coupled to the information processing system. Data from the detection unit may be processed by the processor and data stored in memory. Data on emission profiles for various raman labels may also be stored in memory. The processor may compare the emission spectra from the sample in the flow path and/or flow-through cell to identify the raman-active organic compound. The processor may analyze the data from the detection unit to determine, for example, the sequence of a polynucleotide bound by a silver colloid employed by the methods of the present invention. The information processing system may also perform standard procedures such as subtraction of background signals

While certain methods of the present invention may be performed under the control of a programmed processor, in alternative embodiments of the invention, the methods may be fully or partially implemented by any programmable or hardcoded logic, such as Field Programmable Gate Arrays (FPGAs), TTL logic, or Application Specific Integrated Circuits (ASICs). Additionally, the disclosed methods may be performed by any combination of programmed general purpose computer components and/or custom hardware components.

Following the data gathering operation, the data will typically be reported to a data analysis operation. To facilitate the analysis operation, the data obtained by the detection unit will typically be analyzed using a digital computer such as that described above. Typically, the computer will be appropriately programmed for receipt and storage of the data from the detection unit as well as for analysis and reporting of the data gathered.

In certain embodiments of the invention, custom designed software packages may be used to analyze the data obtained from the detection unit. In alternative embodiments of the invention, data analysis may be performed, using an information processing system and publicly available software packages.

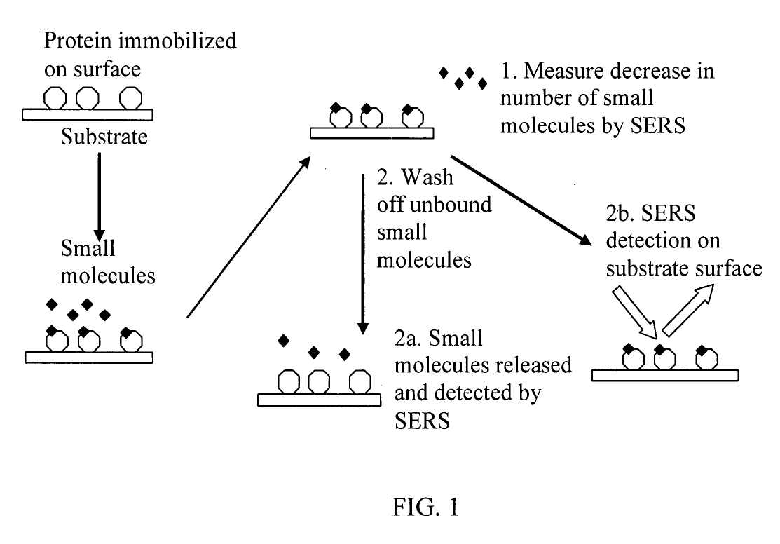

The invention will be further understood with reference to the following FIGS. 1 to 3, which describe exemplary examples, and should not be taken as limiting the true scope of the present invention as described in the claims. All of the methods and tools described with respect to FIGS. 1 to 3 below can be used without any chemical modifications to the analytes being studied. FIG. 1 shows a schematic of an embodiment for detecting the binding of small molecules to proteins immobilized on a substrate surface. In FIG. 1, protein molecules are immobilized on the surface of a substrate. A variety of substrates can be used, including glass slides, flat metal surfaces and metal beads. Although this figure shows the detection of small molecules to proteins bound to a substrate surface, the same procedure and devices may be used to detect the binding of a variety of analytes to one another in which one of the analytes is a capture molecule as defined herein.

In FIG. 1 bi-functional linker groups can be used to help bind the protein molecules to the surface of the substrate. Once the protein has been immobilized on the surface of the substrate, the proteins are incubated in a fluid containing a small molecule analyte. Preferably, the small molecules are dissolved in a solution.

If there is a strong binding affinity between the small molecule and the protein, a decrease in the number of free molecules in the fluid can be detected by Raman spectroscopy as shown in FIG. 1-1.

One example of how Raman spectroscopy can be used to detect the decrease in the number of free molecules in the fluid is by first taking a Raman spectrum of the small molecules in the fluid prior to contacting the fluid containing the small molecules to the substrate containing the proteins. The Raman spectrum of the small molecules in the fluid can be enhanced by mixing the fluid sample can with SERS active particles, such as colloidal Ag. A chemical enhancing agent such as Lithium Chloride can then be added to the sample to induce aggregation of the colloidal particles and achieve large enhancement of the Raman spectrum attributable to the small molecules in the fluid. Once the Raman spectrum of the small molecules in the fluid is obtained utilizing known techniques, the fluid is exposed to proteins immobilized on the surface of a substrate.

The fluid is then contacted to the substrate for an adequate period of time and then extracted from the substrate. Another Raman spectrum is obtained from the small molecules in the fluid. If the Raman spectrum of the small molecules in the fluid decreases in intensity or ceases to exist after the fluid has been in contact with the substrate, this can indicate that there is affinity between the small molecule and the protein as some of the small molecules have left the fluid and have bonded to the proteins on the substrate. Conversely, if the Raman spectrum of the small molecules in the fluid does not change in intensity after the fluid has been in contact with the substrate, this can indicate that there is not affinity between the small molecule and the protein. Preferably, SERS active particles are used to enhance the Raman spectrum of the small molecules in the fluid.

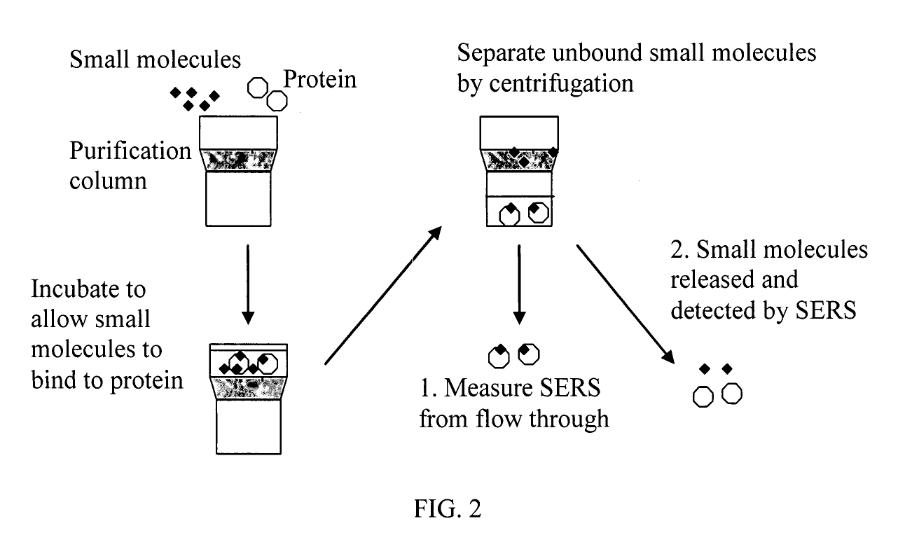

FIGS. 1-2a shows an alternative embodiment for detecting bonding of the small molecules to the protein. In FIG. 1-2(a) the fluid containing the small molecules is again contacted with the substrate containing the proteins. The substrate is then removed from the fluid containing the small molecules and placed into a new environment. The new environment preferably does not contain any of the small molecules.

The substrate is then subjected to conditions for releasing any small molecules attached to the proteins on the substrate. These conditions can include heating the substrate, denaturing the proteins by, for example, sodium dodecyl sulphate (SDS), or replacing the small molecules with molecules that are known to have strong binding affinity to the protein. The environment is then tested for the presence of small molecules by Raman spectroscopy. Again, preferably, SERS active particles are used to enhance the Raman spectrum of the small molecules in the fluid. The SERS active particles, for example, can be in the environment that the substrate is transferred to after being exposed to the fluid.

FIGS. 1-2b shows yet another alternative embodiment for detecting bonding of the small molecules to the protein. In FIGS. 1-2(b) the fluid containing the small molecules is again contacted with the substrate containing the proteins. Raman spectroscopy is then performed directly on the substrate surface itself. If the Raman spectrum that corresponds to the small molecule analyte appears on the surface of the substrate, this can indicate that there has been binding of the small molecule to the protein on the substrate.

In this embodiment preferably the SERS active particles are located on the substrate. Also in this embodiment, preferably the substrate is removed from the fluid containing the small molecules prior to performing the Raman spectroscopy. This may decrease the chance of receiving a Raman spectrum that corresponds to the small molecules in the surrounding fluid instead of on the substrate. Further, the substrate may be washed prior to performing the Raman spectroscopy in order to remove small molecules that have not bonded to the proteins on the substrate surface.

FIG. 2 shows a schematic of an embodiment for detecting the binding of small molecules to proteins in which the proteins are mixed and purified through filtration in a fluid state. In FIG. 2 small molecules and proteins are mixed in a purification column such as a size exclusion spin column and are allowed to incubate for a period of time. The incubation times depends on the binding affinity between the small molecule and the protein. Typically an incubation time of 10-30 minutes at room temperature is sufficient. The solvent used in the purification column is preferably chosen to optimize binding between the small molecules and the proteins. For example, PKA reaction buffer (50 mM Tris-HCl, 10 mM MgCl₂) is preferably used for optimal binding between PKA and H-89.

Small molecules that do not bind to the proteins are then separated from the proteins by centrifugation in the purification column. For example, molecules with molecular weights below a certain cut-off value are retained in the size-exclusion type purification columns while protein molecules are collected in the flow through. Any small molecules that bind to the protein can also be carried through the column with the protein and recovered in the flow through. Raman spectroscopy using SERS active particles can be used to detect the small molecules in the flow through.

In FIG. 2-1 Raman spectroscopy is used to detect the presence of the small molecules while they are attached to the proteins. Preferably, SERS active particles (Ag colloids) are aggregated by Lithium Chloride prior to addition of the protein sample. Raman spectra are collected from the mixture of sample and aggregated Ag colloids.

In FIG. 2-2 the small molecules are first released from the proteins in the flow through by, for example, heating the flow through. Raman spectroscopy is then again used to detect the presence of the small molecules in the flow through.

### EXAMPLE

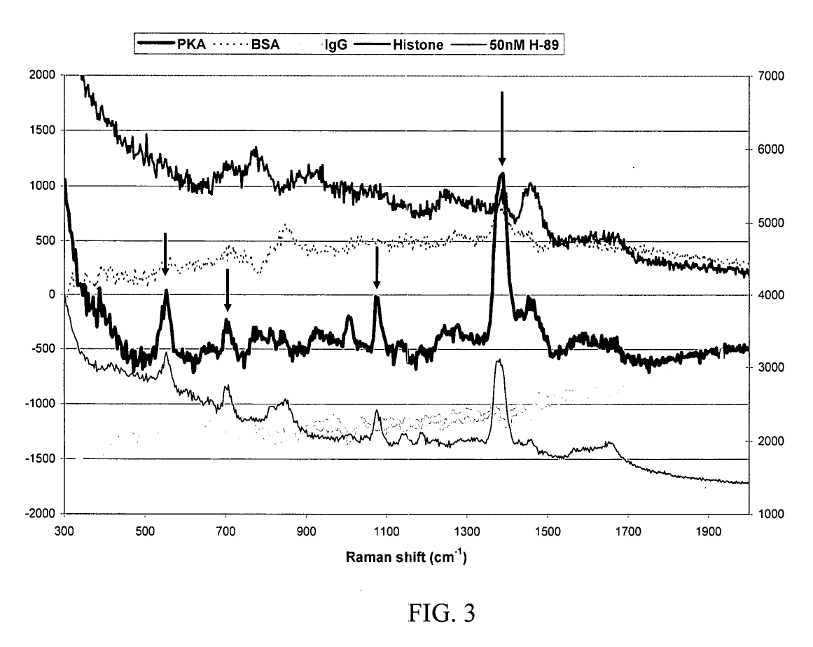

FIG. 3 shows an example of how the binding of small molecules to proteins can be detected according to the process described with respect to FIG. 2.

H-89 is a known small molecule inhibitor to protein kinase A (PKA). The spectrum labeled 50 nM H-89 in FIG. 3 is the Raman signature spectrum of 50 nM H-89 in a H₂O solution (intensity on the secondary y-axis). A silver colloid was used as SERS active particles.

PIERCE ZEBA-ZEBA desalt spin columns (cat #89882) were washed 10 times with methanol and 10 times with purified water. H-89 was then mixed with PKA or control proteins in a PKA reaction buffer (50 mM Tris-HCl, 10 mM MgCl2), making a final H-89 concentration of 2.5 μM and protein concentration of 0.2 mg/mL. This mixture was incubated at room temperature for 30 minutes, added to a desalting size exclusion spin column, and spun at 1500 g for 2 minutes to remove free H-89.

The flow through, which does not contain free H-89, was collected from the column and an aliquot of it is mixed with silver colloids for SERS measurement. The silver colloids are aggregated by 0.5M of Lithium Chloride prior to addition of the sample. Ten spectra are collected from the mixture of sample and aggregated silver colloids. The remaining flow through is heated at 70° C. for 10 minutes, and another SERS measurement is performed. The line labeled PKA is the arithmetic difference in the SERS spectrums at 70° C. and room temperature (intensity on the primary y-axis). The main characteristic peaks of H-89 are visible and marked by black arrows. This indicates that there was binding of H-89 to PKA.

The same procedure described above is repeated with other proteins including bovine serum albumin (labeled BSA), immunoglobulin G (labeled IgG), and Histone (labeled as Histone) as negative controls. The SERS spectrum corresponding to H-89 is not seen in any of these controls. This indicates that specific binding of H-89 to the control proteins did not occur.

Although FIGS. 1-3 describe embodiments in which small molecules and proteins are described as being the analytes. It is understood, however, that the same process and tools can be used to detect the binding of a variety of analytes to one another and the invention is not limited to just the binding of small molecules to proteins.

Commercial applications for the invention methods employing the methods described herein include environmental toxicology and remediation, biomedicine, materials quality control, food and agricultural products monitoring, anaesthetic detection, automobile oil or radiator fluid monitoring, breath alcohol analyzers, hazardous spill identification, explosives detection, fugitive emission identification, medical diagnostics, fish freshness, detection and classification of bacteria and microorganisms both in vitro and in vivo for biomedical uses and medical diagnostic uses, monitoring heavy industrial manufacturing, ambient air monitoring, worker protection, emissions control, product quality testing, leak detection and identification, oil/gas petrochemical applications, combustible gas detection, H₂S monitoring, hazardous leak detection and identification, emergency response and law enforcement applications, illegal substance detection and identification, arson investigation, enclosed space surveying, utility and power applications, emissions monitoring, transformer fault detection, food/beverage/agriculture applications, freshness detection, fruit ripening control, fermentation process monitoring and control applications, flavor composition and identification, product quality and identification, refrigerant and fumigant detection, cosmetic/perfume/fragrance formulation, product quality testing, personal identification, chemical/plastics/pharmaceutical applications, leak detection, solvent recovery effectiveness, perimeter monitoring, product quality testing, hazardous waste site applications, fugitive emission detection and identification, leak detection and identification, perimeter monitoring, transportation, hazardous spill monitoring, refueling operations, shipping container inspection, diesel/gasoline/aviation fuel identification, building/residential natural gas detection, formaldehyde detection, smoke detection, fire detection, automatic ventilation control applications (cooking, smoking, etc.), air intake monitoring, hospital/medical anesthesia & sterilization gas detection, infectious disease detection and breath applications, body fluids analysis, pharmaceutical applications, drug discovery, telesurgery, and the like.

This application discloses several numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1. A method of detecting binding of a first analyte to a second analyte comprising:
contacting a fluid comprising a first analyte to a second analyte attached to a substrate; and
determining whether there is a decrease in the concentration of the first analyte in the fluid after contacting the first analyte in fluid to the second analyte by Raman spectroscopy.

2. The method of claim 1, wherein the first analyte is a molecule with a molecular weight less than 5,000 Da.

3. The method of claim 1, wherein the second analyte is a biomolecule.

4. The method of claim 1, wherein the second analyte is a protein.

5. The method of claim 1, wherein the second analyte is an enzyme.

6. The method of claim 1, wherein the substrate comprises glass, nickel, magnetic metal, gold, silicon, nitrocellulose, or Polyvinylidene Difluoride (PVDF).

7. The method of claim 1, wherein SERS active particles are used in the Raman spectroscopy to enhance a Raman signal of the first analyte.

8. The method of claim 7, wherein the SERS active particles comprise gold, silver, copper, lithium, sodium, potassium, palladium, platinum, or aluminum.

9. The method of claim 1, wherein determining whether there is a decrease in the concentration of the first analyte in the fluid Raman spectroscopy comprises:
obtaining a first Raman spectrum of the first analyte in the fluid prior to contacting the first analyte in the fluid to the second analyte;
obtaining a second Raman spectrum of the first analyte in the fluid after contacting the first analyte in the fluid to the second analyte; and
comparing the first Raman spectrum to the second Raman spectrum.

10. A method of detecting binding of a first analyte to a second analyte comprising:
introducing a second analyte attached to a substrate to an environment that comprises unbound first analyte;
removing the second analyte from the environment comprising unbound first analyte;
subjecting the second analyte to conditions that release the first analyte bound to the second analyte;
detecting the presence of the released first analyte by Raman spectroscopy.

11. The method of claim 10, wherein the conditions that release the first analyte bound to the second analyte comprises heating the substrate, denaturing the second analyte, or replacing the first analyte by competitive binding to the second analyte.

12. The method of claim 10, wherein the detecting the presence of the released first analyte by Raman spectroscopy utilizes SERS active particles to enhance a Raman signal of the first analyte.

13. The method of claim 10, wherein the first analyte is a molecule with a molecular weight less than 5,000 Da.

14. The method of claim 10, wherein the second analyte is a biomolecule.

15. The method of claim 10, wherein the second analyte is a protein.

16. The method of claim 10, wherein the second analyte is an enzyme.

17. The method of claim 10, wherein the substrate comprises glass, nickel, magnetic metal, gold, silicon, nitrocellulose, or Polyvinylidene Difluoride (PVDF).

18. The method of claim 12, wherein the SERS active particles comprise gold, silver, copper, lithium, sodium, potassium, palladium, platinum, or aluminum.

19. A method of detecting binding of a first analyte to a second analyte comprising:
introducing a second analyte attached to a substrate to an environment that comprises a first analyte unbound to the second analyte; and
detecting the presence of the first analyte bound to the second analyte on the substrate by Raman spectroscopy.

20. The method of claim 19, further comprising removing the second analyte attached to the substrate from the environment prior to detecting the presence of the first analyte bound to the second analyte.

21. The method of claim 19, wherein the detecting the presence of the first analyte bound to the second analyte by Raman spectroscopy utilizes SERS active particles to enhance a Raman signal of the first analyte.

22. The method of claim 19, wherein the first analyte is a molecule with a molecular weight less than 5,000 Da.

23. The method of claim 19, wherein the second analyte is a biomolecule.

24. The method of claim 19, wherein the second analyte is a protein.

25. The method of claim 19, wherein the second analyte is an enzyme.

26. The method of claim 19, wherein the substrate comprises glass, nickel, magnetic metal, gold, silicon, nitrocellulose, or Polyvinylidene Difluoride (PVDF).

27. The method of claim 21, wherein the SERS active particles comprise gold, silver, copper, lithium, sodium, potassium, palladium, platinum, or aluminum.

28. A device for detecting binding of a first analyte to a second analyte comprising:
a first analyte in contact with a second analyte attached to a substrate; and
a Raman spectrometer, wherein the Raman spectrometer detects binding of the first analyte to the second analyte attached to the substrate.

29. The device of claim 28, wherein the first analyte can be unbound from the second analyte prior to being detected by the Raman spectrometer.

30. The device of claim 28, wherein the Raman spectrometer detects the presence of the first analyte while the first analyte is attached to the second analyte.

31. The device of claim 29, wherein the first analyte can be unbound from the second analyte by heating the substrate, denaturing the second analyte, or replacing the first analyte by competitive binding to the second analyte.

32. The device of claim 28, further comprising SERS active particles.

33. The device of claim 28, wherein the first analyte is a molecule with a molecular weight less than 5,000 Da.

34. The device of claim 28, wherein the second analyte is a biomolecule.

35. The device of claim 28, wherein the second analyte is a protein.

36. The device of claim 28, wherein the second analyte is an enzyme.

37. The device of claim 28, wherein the substrate comprises glass, nickel, magnetic metal, gold, silicon, nitrocellulose, or Polyvinylidene Difluoride (PVDF).

38. The device of claim 32, wherein the SERS active particles comprise gold, silver, copper, lithium, sodium, potassium, palladium, platinum, or aluminum.

39. A method of detecting binding of a first analyte to a second analyte comprising:
contacting a first analyte to a second analyte to form a complex comprising the first analyte bound to the second analyte;
separating unbound first analyte from the complex; and
detecting the presence of the first analyte in the complex by Raman spectroscopy.

40. The method of claim 39, wherein the unbound first analyte is separated from the complex by a process that comprises centrifugation or filtration.

41. The method of claim 39, wherein the contacting the first analyte to the second analyte is performed in a fluid comprising the first analyte and the second analyte.

42. The method of claim 39, wherein the first analyte is a molecule with a molecular weight less than 5,000 Da.

43. The method of claim 39, wherein the second analyte is a biomolecule.

44. The method of claim 39, wherein the second analyte is a protein.

45. The method of claim 39, wherein the second analyte is an enzyme.

46. The method of claim 39, wherein SERS active particles are used to enhance a Raman signal of the first analyte in the complex.

47. The method of claim 39, wherein the SERS active particles comprise gold, silver, copper, lithium, sodium, potassium, palladium, platinum, or aluminum.

48. The method of claim 39, wherein detecting the presence of the first analyte in the complex by Raman spectroscopy comprises separating the first analyte from the complex and detecting the presence of the separated first analyte.

49. A method of detecting binding of a first analyte to a second analyte comprising:
contacting unbound first analyte to a second analyte to form a complex comprising the first analyte bound to the second analyte; and
detecting a decrease in the concentration of the unbound first analyte after contacting the first analyte to the second analyte in the complex by Raman spectroscopy.

50. The method of claim 49, wherein the complex is separated from the unbound first analyte prior to detecting a decrease in the concentration of the unbound first analyte.

51. The method of claim 49, wherein the unbound first analyte is separated from the complex by a process that comprises centrifugation or filtration.

52. The method of claim 49, wherein the contacting the unbound first analyte to the second analyte is performed in a fluid comprising the unbound first analyte and the second analyte.

53. The method of claim 49, wherein the first analyte is a molecule with a molecular weight less than 5,000 Da.

54. The method of claim 49, wherein the second analyte is a biomolecule.

55. The method of claim 49, wherein the second analyte is a protein.

56. The method of claim 49, wherein the second analyte is an enzyme.

57. The method of claim 49, wherein SERS active particles are used in to enhance a Raman signal of the unbound first analyte.

58. The method of claim 57, wherein the SERS active particles comprise gold, silver, copper, lithium, sodium, potassium, palladium, platinum, or aluminum.
